(19) United States
(12) Patent Application Publication
(10) Pub. No.: US 2018/0085001 A1
(43) Pub. Date: Mar. 29, 2018

(54) NEUROPROTECTION APPARATUS

(71) Applicants: Abraham Berger, Givataim (IL); Avri Hazan, Givataim (IL)

(72) Inventors: Abraham Berger, Givataim (IL); Avri Hazan, Givataim (IL)

(21) Appl. No.: 15/564,907

(22) PCT Filed: May 9, 2016
(86) PCT No.: PCT/IL2016/050488

§ 371 Date: Oct. 6, 2017

(30) Foreign Application Priority Data
May 10, 2015 (IL) 238716

(51) Int. Cl. A61B 5/00; A61F 7/12; A61B 5/01; A61B 90/00

(52) U.S. Cl. CPC A61B 5/0042; A61F 7/12; A61B 5/015; A61B 5/4836; A61B 2034/104; A61B 5/7282; A61B 90/04; A61B 5/4064; A61B 5/6803

(57) ABSTRACT

Provided is a neuroprotection apparatus including a scalp temperature measurement acquisition device for acquiring scalp temperature measurements at a plurality of locations on a patient's scalp during an induced hypothermia and a neuroprotection processor for processing the scalp temperature measurements for determining real time patient temperature information for display on a human head image on a display device during the induced hypothermia.

### FIELD OF THE INVENTION

The invention relates to neuroprotection apparatus.

### BACKGROUND OF THE INVENTION

Induced hypothermia is an established therapeutic treatment for treating medical conditions and an established therapeutic procedure during surgical procedures. Induced hypothermia can be classified as either induced systemic hypothermia for controlled reduction of a patient's core body temperature below 37 ° C. or induced local hypothermia for controlled reduction of the temperature of a particular organ or body part below 37° C. Induced hypothermia classification delineates induced mild hypothermia at 34-36.9° C., induced moderate hypothermia at 32-33.9° C., inducted moderate/deep hypothermia at 30-31.9° C. and deep induced hypothermia at <30° C. Conventional techniques for achieving induced systemic hypothermia include inter alia entire body immersion in ice water to reduce core body temperatures, a heart-lung machine to cool a patient's blood during open heart surgery, and the like. Heart-lung machines are typically capable of cooling a patient's blood supply from a normal 37° C. core body temperature to a chilled 33° C. Conventional techniques for inducing local brain hypothermia include inter alia helmets for external cooling of a stroke patient's head, IntraArterial Cold Infusion (IACI) regimes, and the like.

A human brain weighs only a small fraction typically in the range of 2-3% of human body weight but human cerebral blood flow is typically about 15% of human cardiac output. Accordingly, the temperature of a human brain changes considerably faster than core body temperature and IACI regimes have been shown to induce highly selective brain temperature decrease within minutes and reach targeted local hypothermia 10 to 30 times faster than conventional induced systemic hypothermia. IACI regimes typically chill a human brain from core body temperature to between 32° C. to 34° C. within 10 minutes.

Terminating induced systemic or local brain hypothermia can lead to neurodamage because of brain tissue heating up too rapidly from its induced hypothermic state. The preferred warming rate of a human brain from a hypothermic state to normal core body temperature is 30 about 0.5° C. per hour. After administration of cold infusion to induce a human brain into a hypothermic state and termination of the cold infusion, a hypothermic human brain naturally warms at a rate of about 2° C. per hour from its hypothermic state to its normal core body temperature due to incoming blood flow at normal 37° C. body core temperature and its ongoing cerebral activity which is a much higher than the preferred 0.5° C. per hour warming rate. Terminating induced systemic hypothermia results in a hypothermic human brain naturally warming at a slower rate than 2° C. per hour but still higher than the preferred 0.5° C. per hour warming rate.

Ischemic strokes lead to the formation of an ischemic infarct as a result of an obstruction by embolism or thrombosis, a hyperemic borderzone upstream of the infarct due to accumulation of vasoactive metabolites and acidosis, and a collaterally perfused ischemic penumbra region downstream of the infarct in the direction of flow of the blood stream. Such obstructions can be from about 70% occlusion and greater. A penumbra region is typically defined when blood perfusion drops below about 20 ml/100 g/min as opposed to normal perfusion of about 50 to 60 ml/100 g/min. The ischemic infarct is irreversibly injured brain tissue whilst the penumbra region is at risk but is salvageable if reperfusion occurs within a reasonably short period of several hours. Salvation of a penumbra region can be assisted by induced either systemic or local brain cooling which attributes to a reduction in oxygen demand: a 1° C. decrease in brain temperature lowers cerebral oxygen consumption by about 5% thus increasing tolerance to ischemic conditions.

IACIs are typically 3° C. to 4° C. saline solutions at a proximal indwelling catheter end 20 before infusion. IACIs are possibly mediated by suitable drugs including inter alia TPA, and the like. Typical IACI regimes involve the infusion of 5 to 30 cc/min over the period of several hours leading to a total saline infusion of from about 2 liter to 3 liter. Clinical practitioners intending on administering an IACI regime to a stroke patient for neuroprotection from ischemic brain damage are required to determine the most suitable cerebral target location of an indwelling catheter. Conventional brain scan technologies including inter alia MRI, CT, and the like, detect the three dimensional boundaries of an ischemic infarct and its associated penumbra for the purpose of accurate placement of an indwelling catheter upstream of an ischemic infarct. The brain scans also include information regarding degrees of occlusions of arteries and/or veins, blood flow in vessels, and perfusion in neurons. Clinical practitioners using conventional fluoroscopy units are capable of relatively accurate placing an indwelling catheter at a desired location. However, an arterial obstruction can cause the diversion of a considerable proportion of infusion flow from its intended artery to an adjoining artery thereby not cooling a penumbra downstream of an occlusion. Moreover, an indwelling catheter also may cause a considerable change to a flow regime of incoming saline due its relative large size compared to a brain artery in which it is placed. Accordingly, it is difficult to predict to what degree a penumbra is being cooled by a chilled infusion flow.

ThermopeutiX, Inc., San Diego, USA is developing systems and methods for selective cooling of a target site including a catheter having a supply lumen and a delivery lumen with inlet and outlet ports. Blood is withdrawn from the supply lumen and cooled or heated in a control unit. The treated blood is sent to a targeted area via delivery lumen. ThermopeutiX, Inc. is assignee of inter alia U.S. Pat. No. 8,192,392 entitled Methods for Selective Thermal Treatment to Solar et al.

### Neuroprotection Apparatus and Method Disclosures Include Inter Alia

“Brain temperature changes during selective cooling with endovascular Intracarotid Cold Saline Infusion (ICSI): simulation using human data fitted with an integrated mathematical model”, published in Journal of NeuroIntervention Surgery 2013; 5:165-171 by Matthew Aaron Harold Neimark et al. discloses a 3D thermal model for determining a temperature at spherical sections in a 3D space. The article discloses the use of dominant superior internal Jugular Venous Bulb blood Temperature (JVBT) as a surrogate for ipsilateral brain temperature. The article discloses the 3D thermal model is applied in two modes: (1) a forward model mode runs the 3D thermal model using initial conditions particular to each patient; and (2) an inverse model mode fits measured JVBT data to the model. The output of the forward mode provides expected jugular venous temperatures which may be used as a rough means of validating model fidelity. The output of the inverse mode provides estimates of intracranial brain temperatures which may be used as a means to better evaluate the feasibility of ICSI to obtain therapeutic brain temperatures. The article discusses that jugular venous blood contains a mixture of blood draining superficial and deep brain tissue from both cerebral hemispheres, infratentorial brain and extracranial tissues such that more accurate determination of the actual brain or Ipsilateral Anterior Circulation Territory (IACT) temperature during Intracarotid Cold Saline Infusion (ICSI) would be beneficial.

U.S. Pat. No. 8,343,097 entitled Systems and Methods for Intravascular Cooling to Pile-Spellman et al. discloses an indwelling catheter with a multitude of sensors therealong and a feedback system utilized to control the volume, temperature and/or infusate rate of an infusate to achieve a predetermined temperature at a target location based on sensed temperatures. US '097 FIG. 8 shows a guide catheter 80 for guiding a micro-catheter 86 for delivery of infusate. Longitudinally adjacent to the micro-catheter 86 is a wire 92 having a sensor 96 to measure a parameter of the infusate and blood mixture, a sensor 98 to measure a parameter e.g. the temperature of the infusate at a distal end 88 of the micro-catheter 86, a sensor 100 for measuring reflux, and a sensor 102 positioned to measure a parameter of the patient's blood, for example, the patient's core body temperature.

There is a need for neuroprotection apparatus for assisting clinical practitioners to monitor brain temperatures during three phases of induced either systemic or local brain hypothermia as follows: Phase 1 for cooling of a human brain from normal 37° C. body core temperature to steady state hypothermia. Phase 2 for maintaining a human brain at steady state hypothermia. And Phase 3 for warming a human brain from steady state hypothermia to normal 37° C. body core temperature. There is yet a further need for improved administration of Intra-Arterial Cold Infusion (IACI) regimes for inducing local brain hypothermia.

### SUMMARY OF THE INVENTION

The present invention is directed towards neuroprotection apparatus including a scalp temperature measurement acquisition device for acquiring scalp temperature measurements at a plurality of locations on a patient's scalp during an induced hypothermia and a neuroprotection processor for processing the scalp temperature measurements for determining real time patient temperature information for display on a human head image on a display device during the induced hypothermia.

Cerebral temperature changes take at least several minutes on infusion of a chilled infusion liquid to consecutively chill a brain, a cranium and a scalp to induce detectable temperature changes on a patient's scalp such that neuroprotection apparatus of the present invention affords near real-time clinical feedback. Moreover, cerebral temperature changes are also a function of the locations of one or more cold infusion sources in a patient's head. Accordingly, in the case of a single cold infusion source deployed in a patient's right middle cerebral artery (MCA), it will take longer for a detectable temperature change to appear on the left side of the patient's head as opposed to his right side. Also, after reaching hypothermic steady state, the right side of the patient's head will be colder than his left side.

The neuroprotection processor can process acquired scalp temperature measurements for determining a so-called patient scalp isotherm map. The scalp temperature measurements can be the result of induced either systemic or local brain hypothermia. Patient scalp isotherm maps typically include at least two scalp isotherms, namely, an innermost scalp isotherm which is the coldest scalp isotherm and an outermost scalp isotherm which is the warmest scalp isotherm corresponding to a normal scalp skin temperature typically in the range of from 34° C. to 35° C. depending on ambient temperatures. The human head image can preferably be an image of a patient's head.

The neuroprotection processor can process thermodynamic information in a 3D thermal model of an upper generally hemispherical section of a human head including its brain, cranium and scalp, for determining a so-called patient 3D temperature estimation mapping. The patient 3D temperature estimation mapping includes temperature estimations of 3D finite elements within the upper generally hemispherical section of a human head as bounded by the 3D thermal model. A suitable 3D thermal model is described in hitherto mentioned “Brain temperature changes during selective cooling with endovascular intracarotid cold saline infusion: simulation using human data fitted with an integrated mathematical model”, Neimark et al., published in J NeuroIntervent Surg 2013:5:1651-71 doi:10.1136/neurintsurg-2011-010150.

The 3D thermal model can be applied with induced either systemic or local hypothermia. In induced systemic hypothermia, the 3D thermal model models four cold infusion sources delivering chilled blood to a human brain, namely, a right pair of a carotid artery and a vertebral artery and a left pair of a carotid artery and a vertebral artery. In induced local brain hypothermia, the 3D thermal model typically models a single indwelling catheter tip as a cold 25 infusion source delivering infusion liquid to a human brain. The infusion liquid delivered by the single indwelling catheter may be saline, blood, a combined blood saline infusion, and the like. The 3D thermal model processes flow parameters associated with a cold infusion source including inter alia infusion temperature, infusion flow rate, and the like. The flow parameters can be obtained from a suitable indwelling catheter fitted with required sensors at its catheter tip. Also, the neuroprotection processor processes the location of the catheter tip of an indwelling catheter. The neuroprotection processor can display the temperature estimations of the 3D finite elements on one or more of the standard sagittal planes, transverse planes and coronal planes, and also cross sections selected by a clinical practitioner. The neuroprotection processor preferably displays the 3D temperature estimation mapping of the 3D finite elements on a MRI/CT scan image of a patient's head.

One preferred embodiment of a scalp temperature measurement acquisition device in accordance with the present invention includes one or more thermal imaging cameras for acquiring scalp temperature measurements. Another preferred embodiment of a scalp temperature measurement acquisition device in accordance with the present invention includes a temperature measurement sensor array in skin contact with a patient's scalp for acquiring scalp 10 temperature measurements. The scalp temperature measurement acquisition device can include a scalp cooling arrangement for external cooling a patient's scalp for assisting induced local hypothermia of stroke brains. The scalp temperature measurement acquisition devices can be designed as a disposable medical grade item intended for single patient use. Alternatively, scalp temperature measurement acquisition devices can be intended for multiple patient use. Single patient use scalp temperature measurement acquisition devices with a temperature measurement sensor array in skin contact with a patient's scalp can resemble stretch fit swim caps.

The neuroprotection apparatus can include a computer database having a benchmark stroke brain clinical image collection of benchmark stroke brain clinical images. The benchmark stroke brain clinical images each include an occlusion, an ischemic infarct and a downstream penumbra region. The benchmark stroke brain clinical images differ in terms of the location of an ischemic infarct, an occlusion percentage and size of its associated penumbra region. The benchmark stroke brain clinical images preferably also include clinical instructions including at least one of a placement of an indwelling catheter, infusion liquid flow rate, infusion liquid temperature and infusion liquid duration for administrating infusion liquid. A clinical practitioner can refer to the benchmark stroke brain clinical image collection to assist him to decide the most suitable IACI regime for a particular stroke patient.

The computer database can also include benchmark temperature information for assisting a clinical practitioner to monitor an induced either systemic or local brain hypothermia. The benchmark temperature information can assist a clinical practitioner to decide whether he needs to adjust one or more of the location of an indwelling catheter, an infusion liquid temperature and an infusion liquid flow rate during administration of an IACI regime thereby improving their neuroprotection efficacy. The benchmark temperature information can assist a clinical practitioner during the three phases of an induced either systemic or local brain hypothermia. The benchmark temperature information can include a benchmark scalp isotherm map collection of benchmark scalp isotherm maps and/or a benchmark 3D temperature estimation mapping collection of benchmark 3D temperature estimation mappings. The benchmark scalp isotherm map collection of benchmark scalp isotherm maps are cross referenced to the benchmark stroke brain clinical image collection of benchmark stroke brain clinical images. Similarly, the benchmark 3D temperature estimation mapping collection of benchmark 3D temperature estimation mappings are cross referenced to the benchmark stroke brain clinical image collection of benchmark stroke brain clinical images.

The neuroprotection processor can be programmed to compare acquired patient scalp temperature measurements to corresponding benchmark temperature information during an ongoing induced systemic or local brain hypothermia to determine a benchmark error index. Benchmark index errors can be useful for indicating a range of clinical occurrences requiring corrective action. Exemplary clinical occurrences include inter alia an incorrect region of a human brain is being cooled, a human brain is being chilled to too low a hypothermic temperature, a human brain is warming at a higher rate than the preferred 0.5° C. warming rate, and the like. The neuroprotection processor can compare the benchmark error index to a benchmark error threshold to automatically issue an alert for alerting a clinical practitioner.

### BRIEF DESCRIPTION OF DRAWINGS

In order to understand the invention and to see how it can be carried out in practice, preferred embodiments will now be described, by way of non-limiting examples only, with reference to the accompanying drawings in which similar parts are likewise numbered, and in which:

FIG. 1 is a schematic diagram of a human head of a stroke patient with an ischemic infarct and a downstream penumbra region;

FIG. 2 is a schematic diagram of neuroprotection apparatus for displaying a patient scalp isotherm map on a human head image during administration of an IACI regime;

FIGS. 3A to 3D are schematic diagrams showing progressive cooling of a stroke patient's scalp during administration of an IACI regime from its inception to steady state hypothermia;

FIG. 4 is a schematic diagram of neuroprotection apparatus for displaying a patient scalp isotherm map and a patient 3D temperature estimation mapping on a human head image during administration of an IACI regime;

FIGS. 5A to 5D are schematic diagrams showing the FIG. 4's patient 3D temperature estimation mapping and three different transverse cross sections;

FIG. 6 is a schematic diagram of neuroprotection apparatus for displaying a patient scalp isotherm map and a patient 3D temperature estimation mapping of a patient in steady state hypothermia after induced systemic hypothermia on a human head image;

FIG. 7 is a schematic diagram of a first embodiment of a scalp temperature measurement acquisition device of the neuroprotection apparatus;

FIG. 8 is a schematic diagram of a second embodiment of a scalp temperature measurement acquisition device of the neuroprotection apparatus;

FIG. 9 is a schematic diagram of a third embodiment of a scalp temperature measurement acquisition device of the neuroprotection apparatus resembling a stretch fit swim cap;

FIG. 10 is a rear view of a patient wearing the FIG. 9 scalp temperature measurement acquisition device;

FIG. 11 is a schematic diagram of a clinical set up for diagnosis of a stroke patient;

FIG. 12 is a schematic diagram of a clinical set up for placing an indwelling catheter at a cerebral target location of a stroke brain;

FIG. 13A is a schematic clinical image along a coronal cross section of a non-stroke brain showing a placement of an indwelling catheter at a cerebral target location;

FIG. 13B is a schematic diagram showing a human head image and a patient scalp isotherm map resulting from the FIG. 13A placement of the indwelling catheter and a 15 cc/min flow rate of 12° C. saline infusion at hypothermic steady state;

FIG. 13C is a schematic diagram showing a human head image and a patient scalp isotherm map resulting from the FIG. 13A placement of the indwelling catheter and a 10 cc/min flow rate of 12° C. saline infusion at hypothermic steady state;

FIG. 13D is a schematic diagram showing a human head image and a patient scalp isotherm map resulting from the FIG. 13A placement of the indwelling catheter and a 10 cc/min flow rate of 18° C. saline infusion at hypothermic steady state;

FIG. 14A is a schematic clinical image along a coronal cross section of a stroke brain having a 70% occlusion and a placement of an indwelling catheter for inducing local hypothermia in its associated penumbra region;

FIG. 14B is a schematic diagram showing a s patient calp isotherm map resulting from the FIG. 14A placement of the indwelling catheter on a human head image at hypothermic steady state;

FIG. 15A is a schematic clinical image along a coronal cross section of a stroke brain having a 70% occlusion and a placement of an indwelling catheter for inducing local hypothermia in its associated penumbra region;

FIG. 15B is a schematic diagram showing a patient scalp isotherm map resulting from the FIG. 15A placement of the indwelling catheter on a human head image at hypothermic steady state;

FIG. 16A is a schematic clinical image along a coronal cross section of a stroke brain having a 90% occlusion and a placement of an indwelling catheter for inducing local hypothermia in its associated penumbra region;

FIG. 16B is a schematic diagram showing a patient scalp isotherm map resulting from the FIG. 16A placement of the indwelling catheter on a human head image at hypothermic steady state;

FIG. 17A is a schematic clinical image along a coronal cross section of a stroke brain having a 90% occlusion and showing the use of a micro-catheter for traversing the 90% occlusion for delivering cold saline downstream to its associated penumbra region;

FIG. 17B is a schematic diagram showing a patient scalp isotherm map resulting from the 25 FIG. 17A placement of the indwelling catheter on a human head image at hypothermic steady state; and

FIG. 18 is a flow diagram of a method of administering an IACI regime to a stroke brain.

### DETAILED DESCRIPTION OF PREFERRED EMBODIMENTS OF THE INVENTION

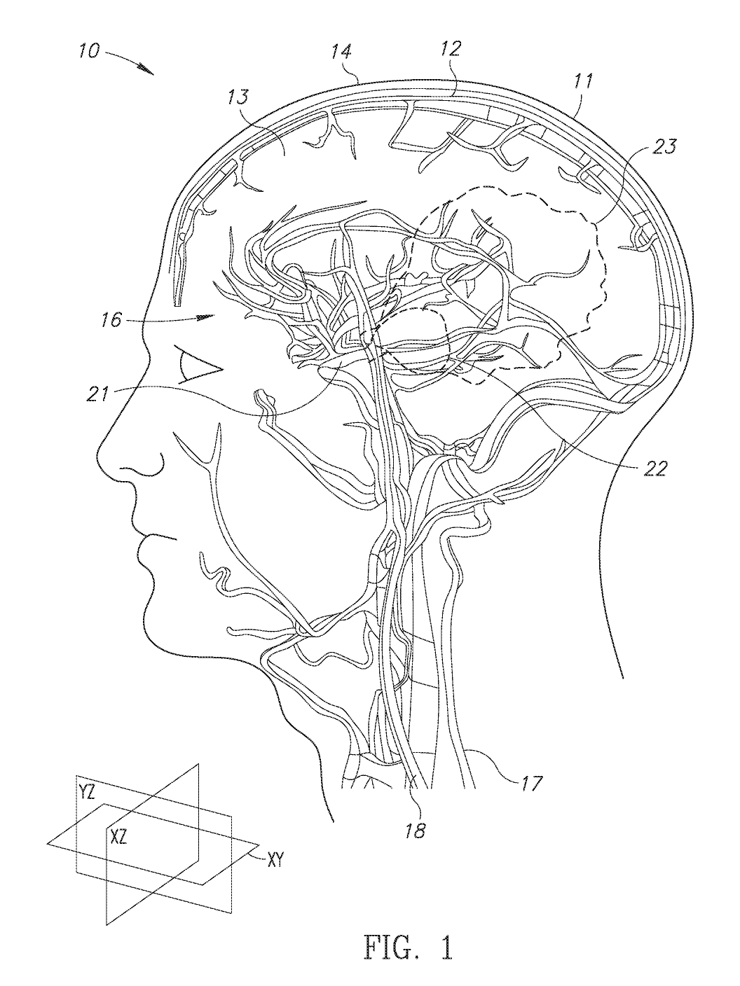

FIG. 1 shows a patient head 10 including a scalp 11, a cranium 12 and a brain 13. Scalps 11 are typically described as having five layers including an outermost scalp skin surface 14. At standard ambient temperature of 25° C., the normal outermost scalp skin surface temperature Ts is between about 34° C. and about 35° C. which is less than 37° C. core body temperature. The brain 13 includes a cerebral circulatory structure 16 including a right pair of a carotid artery 17 and a vertebral artery 18. The cerebral circulatory structure 16 similarly includes a left pair of a carotid artery and a vertebral artery (not shown). Each pair of the right pair and left pair of a carotid artery and a vertebral artery deliver a combined blood flow rate of about 350 cc/min.

The patient is a stroke victim with a stroke brain having an occlusion 21 located, for example, along a Middle Cerebral Artery (MCA). The occlusion 21 can typically occupy from about 70% to about 90% of a cross sectional diameter of a cerebral artery. The occlusion 21 leads to an ischemic infarct 22 and a downstream penumbra region 23. FIG. 1 also shows a Cartesian coordinate system including an XY transverse plane, an XZ sagittal plane, and an YZ coronal plane.

### Neuroprotection Apparatus

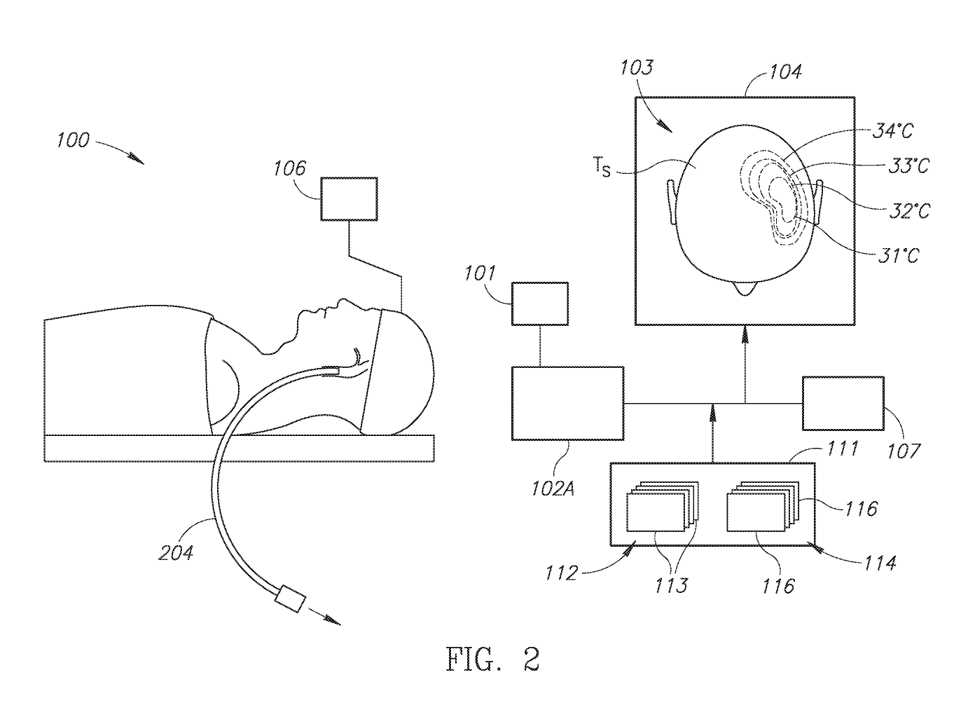

FIG. 2 shows neuroprotection apparatus 100 includes a scalp temperature measurement acquisition device 101 for acquiring scalp temperature measurements at a plurality of locations on a patient scalp during an induced local brain hypothermia and a neuroprotection processor 102A for processing acquired scalp temperature measurements for real time determining a patient scalp isotherm map 103 for display on a human head image on a display device 104 during the induced local brain hypothermia. The neuroprotection processor 102A runs an isotherm determination algorithm for processing acquired scalp temperature measurements for real-time determining the patient scalp isotherm map 103. The patient scalp isotherm map 103 is preferably color coded across a spectrum of colors with blue/green shades representing colder isotherms and red/orange/yellow shades representing warmer isotherms.

FIG. 2 shows the neuroprotection apparatus 100 for determining a patient scalp isotherm map 103 during administration of an IACI regime by way of an indwelling catheter 204 to a stroke patient. FIG. 2 could equally show the use of the neuroprotection apparatus 100 for determining a patient scalp isotherm map during induced systemic hypothermia. The display device 104 can be a computer screen, a wearable display device, and the like. The scalp temperature measurement acquisition device 101 can optionally be provided with a scalp cooling arrangement 106 for external cooling a patient scalp for assisting induced either systemic or local hypothermia.

The neuroprotection apparatus 100 includes an operator interface 107 for entering patient information and induced hypothermia procedure details. Patient details include inter alia name, age, weight, and the like. Induced hypothermia procedure details include inter alia date, time, clinical practitioner, and the like. The operator interface 107 also enables a clinical practitioner to write notes in a free text box.

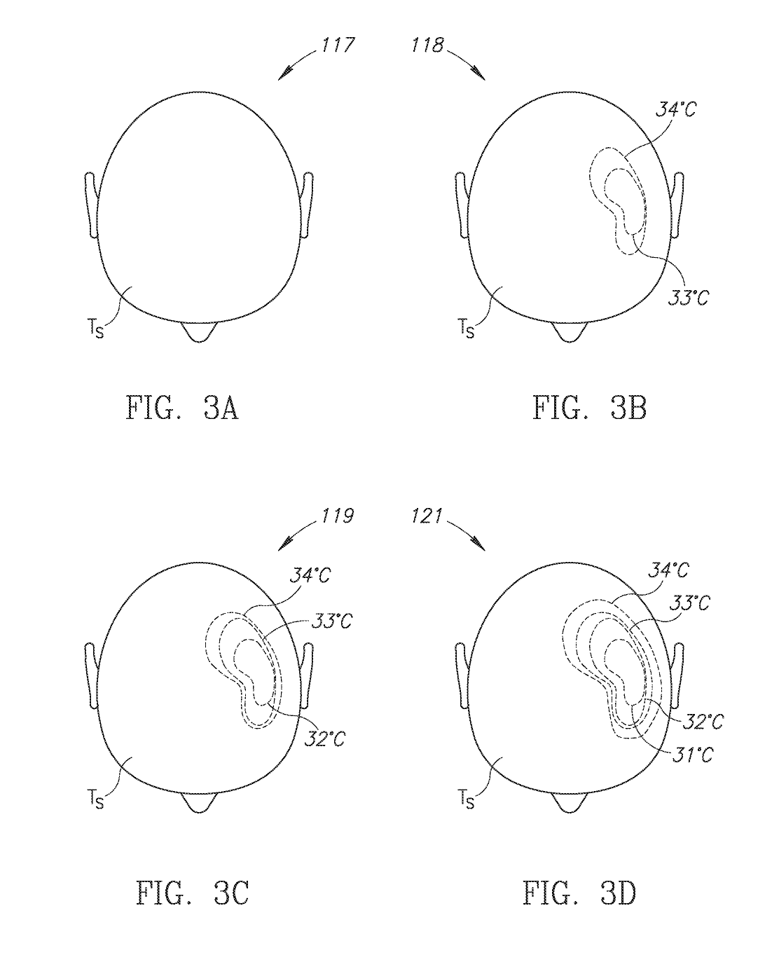

FIGS. 3A to 3D show progressive cooling of a stroke patient's scalp during administration of an IACI regime from its inception at normal scalp skin temperature Ts to hypothermic steady state as evidenced by the development of a cold spot on the stroke patient's scalp. FIG. 3A shows a patient scalp isotherm map 117 including the normal scalp skin temperature Ts before the start of the administration of the IACI regime. FIG. 3B shows a patient scalp isotherm map 118 including an innermost 33° C. scalp isotherm and an outermost 34° C. scalp isotherm after, say, 10 minutes of the administration of the IACI regime. FIG. 3C shows a patient scalp isotherm map 119 including an innermost 32° C. scalp isotherm, an intermediate 33° C. scalp isotherm and an outermost 34° C. scalp isotherm after, say, 20 minutes of the administration of the IACI regime. FIG. 3D shows a patient scalp isotherm map 121 including an innermost 31° C. scalp isotherm, two intermediate 32° C. and 33° C. scalp isotherms and an outermost 34° C. scalp isotherm at hypothermic steady state.

FIGS. 3A to 3D in reverse order, namely, from FIG. 3D to FIG. 3A, show progressive warming of a stroke patient's scalp from hypothermic steady state to normal scalp skin temperature Ts as evidenced by the disappearance of the cold spot from the stroke patient's scalp. Warming of a stroke brain is generally much slower than the cooling of a stroke brain and 25 takes place at the preferred rate of 0.5° C. per hour. Thus, the progressive warming from FIG. 3D to FIG. 3A takes longer than the progressive cooling from FIG. 3A to FIG. 3D.

FIG. 2 shows the neuroprotection apparatus 100 preferably includes a computer database 111 for assisting in a clinical management of induced either systemic or local brain hypothermia. The computer database 111 includes a benchmark stroke brain clinical image collection 112 of benchmark stroke brain clinical images 113. The benchmark stroke brain clinical images 113 are prepared on the analysis of multiple treatments of stroke patients to arrive at optimal efficacy for preventing further infarction of a penumbra region. The benchmark stroke brain clinical images 113 each include an occlusion, an ischemic infarct and a downstream penumbra region. The benchmark stroke brain clinical images 113 differ in terms of a location and percentage of an occlusion, an ischemic infarct, a size and location of an infarct and a size and location of its associated penumbra region.

The benchmark stroke brain clinical images 113 preferably include preferred cerebral target locations for placement of indwelling catheters. The benchmark stroke brain clinical images 113 preferably include instructions regarding a preferred infusion liquid temperature, a preferred infusion liquid flow rate and a preferred infusion administration time for administrating an infusion. The benchmark stroke brain clinical images 113 also preferably include instructions regarding the use of micro-catheters for traversing through nearly 100% occlusions for directly delivering cold infusion liquid to a downstream penumbra region.

The computer database 111 also preferably includes benchmark temperature information in the form of a benchmark scalp isotherm map collection 114 of benchmark scalp isotherm 15 maps 116. A clinical practitioner can use the benchmark scalp isotherm map collection 114 during induced either systemic or local brain hypothermia to assist him to decide whether the patient's brain is being cooled according to a predetermined clinical protocol. Similarly, a clinical practitioner can use the benchmark scalp isotherm map collection 114 during restoration of a patient's brain from an induced hypothermic state to a normal core body temperature to assist him to decide whether the patient's brain is being warmed according to a predetermined clinical protocol to avoid too rapid warming which can inadvertently lead to neurodamage.

The benchmark scalp isotherm map collection 114 can include benchmark scalp isotherm maps 116 at different time intervals during the three phases of induced systemic or local brain hypothermia, namely, from normal core body temperature to steady state hypothermia, maintaining steady state hypothermia and warming from steady state hypothermia to normal core body temperature. Steady state in induced local brain hypothermia is usually reached within 10 to 20 minutes. Steady state in induced systemic hypothermia usually takes considerably longer. Suitable time intervals between consecutive benchmark scalp isotherm maps 116 are, say, between 3 to 5 minutes. The benchmark scalp isotherm map collection 114 can include benchmark scalp isotherm maps 116 acquired during infusions at different infusion liquid temperatures and/or different infusion liquid flow rates. The benchmark scalp isotherm map collection 114 can include benchmark scalp isotherm maps 116 acquired during induced systemic hypothermia at different blood temperatures and/or different blood rates and/or different body weights.

Exemplary uses of the benchmark scalp isotherm maps 116 are as follows: A clinical practitioner notices that a cold spot is developing on a stroke scalp in a location that does not match the location in which it should be developing in order to best treat a stroke patient. The clinical practitioner decides to reposition the catheter tip of an indwelling catheter to correspondingly modify the location of the developing cold spot.

A clinical practitioner notices that a cold spot is developing on a stroke scalp in a location that does match the location in which it should be developing but is developing too slowly. The clinical practitioner decides to not to reposition the indwelling catheter's catheter tip but rather decrease the infusion liquid temperature being administered at the same infusion liquid flow rate. Alternatively, the clinical practitioner could decide to increase the infusion liquid flow rate at the same infusion liquid temperature. Alternatively, again the clinical practitioner could decide to modify both the infusion liquid flow rate and the infusion liquid temperature.

A clinical practitioner regards that a patient's brain is being warmed too quickly on restoration from steady state hypothermia back to normal body core temperature. The clinical practitioner decides he decreased the infusion liquid flow rate too much and decides to increase same to slow down the warming. Alternatively, the clinical practitioner decides he increased the infusion liquid temperature too much and decides to decrease same to slow down the warming.

The neuroprotection processor 102A can be programmed to superimpose a patient scalp isotherm map on a benchmark scalp isotherm map to assist a clinical practitioner to discriminate the difference therebetween. The neuroprotection processor 102A can be programmed to compare acquired scalp temperature measurements to corresponding benchmark scalp temperature measurements during an ongoing induced systemic or local brain hypothermia to determine a benchmark error index. The benchmark error index can be determined using well known statistical techniques for comparing two sets of actual values and benchmark values. The benchmark error index can possibly include weighting factors for different isotherms, for example, the coldest isotherm can be weighted higher than the warmest isotherm. The neuroprotection processor 102A can compare the benchmark error index to a benchmark error threshold to automatically issue an alert for alerting a clinical practitioner. Suitable alerts include inter alia a visual alert, an audible alert, a SMS, and the like.

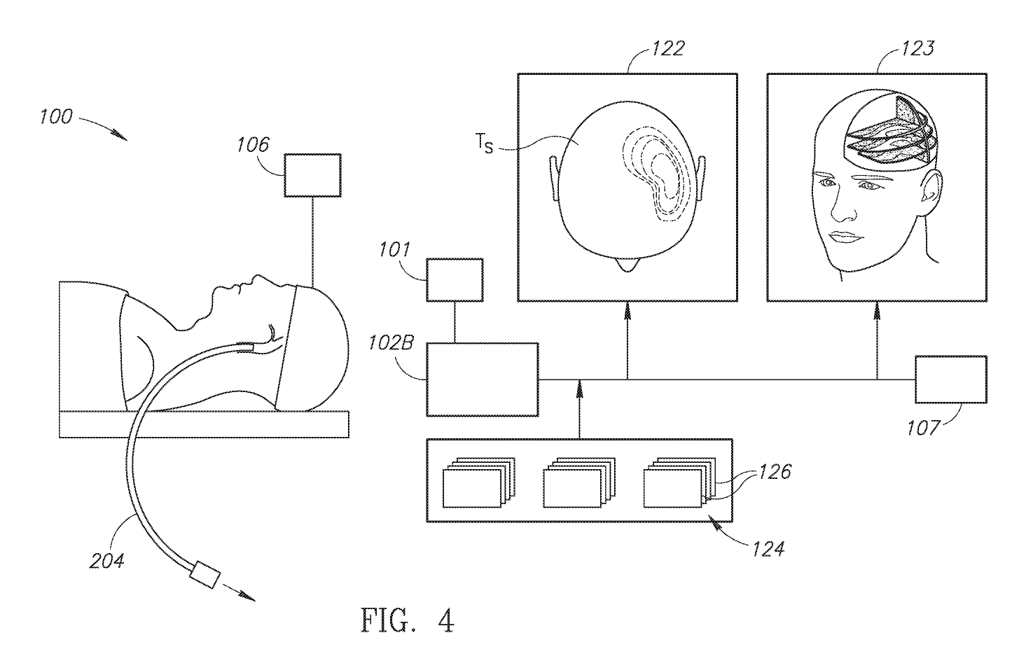

FIG. 4 is a schematic diagram of neuroprotection apparatus 100 during administration of an IACI regime with an indwelling catheter 204. The neuroprotection apparatus 100 includes a neuroprotection processor 102B for real-time determining a patient scalp isotherm map 122 and/or a patient 3D temperature estimation mapping 123 for display on a human head image on a display device. The patient scalp isotherm map 122 can be determined in a similar manner as the neuroprotection processor 102A or derived from the patient 3D temperature estimation mapping 123.

The neuroprotection processor 102B runs a 3D thermal model of an upper generally hemispherical section of a human head including its brain, cranium and scalp, for determining the patient 3D temperature estimation mapping 123. A suitable 3D thermal model can be based on a mathematical model described in hitherto described “Brain temperature changes during selective cooling with endovascular intracarotid cold saline infusion: simulation using human data fitted with an integrated mathematical model”, Neimark et al., published in J NeuroIntervent Surg 2013:5:1651-71 doi:10.1136/neurintsurg-2011-010150. The patient 3D temperature estimation mapping 123 includes temperature estimations of 3D finite elements of the 3D thermal model. The 3D finite elements of the 3D thermal model can be color coded in a similar manner to the scalp isotherm map 103.

Human brains have similar but not identical structures such that a 3D thermal model can only model an exemplary human brain and not a particular patient brain. The location of a placement of an indwelling catheter's catheter tip can only be estimated to within a few millimeters but such few millimeters can lead to considerable changes in cerebral blood flow. Also, the presence of an indwelling catheter and an infarct within an artery can considerably modify cerebral blood flow and/or cold infusion liquid flow within a stroke brain. Accordingly, the neuroprotection processor 102B runs the 3D thermal model in two modes as follows: a 3D thermal model configuration mode and a configured 3D thermal model ongoing procedure mode. The 3D thermal model configuration mode is intended to configure certain parameters of the 3D thermal model to a particular induced systemic or local brain hypothermia at hand including determining the location of an indwelling catheter's catheter tip within the 3D thermal model. The configured 3D thermal model ongoing procedure mode runs the configured 3D thermal model for the remainder of a patient induced hypothermia procedure.

### 3D Thermal Model Configuration Mode

The neuroprotection processor 102B processes flow parameters associated with an infusion source including inter alia infusion temperature, infusion flow rate, infusion duration, and the like, and acquired scalp temperature measurements. The flow parameters can be obtained from a suitable indwelling catheter fitted with required sensors at its catheter tip. The neuroprotection processor 102B also processes the location of a catheter tip of an indwelling catheter within a human brain. The flow parameters can be input manually at the operator interface 107. Alternatively, the flow parameters can be input to the neuroprotection processor 102B automatically. For example, the location of a catheter tip can be determined by a magnetic sensor.

The neuroprotection processor 102B can run the 3D thermal model in a forward model mode only in an iterative closed loop manner as follows: The neuroprotection processor 102B processes flow parameters of an induced hypothermia including the placement of an indwelling catheter, the infusion liquid flow rate and the infusion liquid temperature for determining a patient 3D temperature estimation mapping of a upper generally hemispherical section of a patient's head. The neuroprotection processor 102B compares the 3D temperature estimations at particular locations on the patient's scalp to the acquired scalp temperature measurements thereat to determine the deviations therebetween. The neuroprotection processor 102B can calculate a deviation between the 3D temperature estimation at a particular scalp location and the acquired scalp temperature measurement thereat from at least tens of scalp locations up to thousands of scalp locations depending on the scalp temperature measurement acquisition device 101. The greater the number of scalp locations taken into account improves the accuracy of the neuroprotection processor 102B but with the downside that each run of the 3D thermal model takes longer.

The neuroprotection processor 102B determines a deviation index based on the calculated deviations. The deviation index can be determined using well known statistical techniques for comparing two sets of actual values and estimated values. The deviation index can possibly include weighting factors for different isotherms, for example, the coldest isotherm can be weighted higher than the warmest isotherm. The neuroprotection processor 102B compares the calculated deviation index to a deviation threshold for determining whether to re-position the placement of the indwelling catheter in the 3D thermal model. For example, a deviation index can be the average temperature difference at all the scalp locations at which actual scalp temperature measurements have been acquired and a deviation threshold can be ±0.5° C. temperature difference. The deviation threshold can be optionally set by a clinical practitioner by means of the operator interface 107.

The neuroprotection processor 102B configures the 3D thermal model in the case a deviation index is less than a deviation threshold. If, however, a deviation index is greater than a deviation threshold, the neuroprotection processor 102B modifies the location of the placement of the indwelling parameter within the 3D thermal model and re-runs the 3D thermal model. The location of the placement of the indwelling catheter is selected from the three flow parameters for remodeling purposes because the infusion liquid flow rate and the infusion liquid temperature can be measured to a much higher degree of accuracy than the location of the placement of the indwelling catheter can be estimated based on a fluoroscopy image.

The direction of modification of the location of the placement of an indwelling catheter within an artery depends on whether the scalp temperature estimations are at higher temperatures than the actual scalp measurement temperatures or at lower temperatures than the actual scalp measurement temperatures. In the case of a particular scalp location, if the scalp temperature estimation thereat is colder than the acquired scalp temperature measurement thereat, then the 3D thermal model assumed the placement of an indwelling catheter is further downstream along an artery than it actually is on running the 3D thermal model. Therefore, the 3D thermal model needs to be re-run with the indwelling catheter placed further upstream. If, however, the scalp temperature estimation is warmer than the acquired scalp temperature measurement thereat, then the 3D thermal model assumed a placement of an indwelling catheter is more upstream along an artery than it actually is on running the 3D thermal model. Therefore, the 3D thermal model needs to be re-run with the indwelling catheter placed further downstream.

Alternatively, the neuroprotection processor 102B can run the 3D thermal model in the same two model modes as the hitherto described Neimark article, namely, in a forward model mode and an inverse model mode. In the forward model mode, the neuroprotection processor 102B processes flow parameters associated with an infusion source including inter alia infusion temperature, infusion flow rate, and its location. In the inverse model mode, the neuroprotection processor 102B processes the acquired scalp temperature measurements.

### Configured 3D Thermal Model Ongoing Procedure Mode

After configuration, the neuroprotection processor 102B proceeds to periodically run the configured 3D thermal model to display the patient scalp isotherm map 122 and/or a patient 3D temperature estimation mapping 123 on a human head image on a display device during an ongoing hypothermia. The neuroprotection apparatus 100 continues to acquire scalp temperature measurements from a patient scalp during ongoing procedure mode as a back-up safety measure in view of occurrences which can take occur during induced systemic or local brain hypothermia. Exemplary occurrences can include inter alia movement of an indwelling catheter, change in cerebral blood flow, formation of a blockage along an indwelling catheter, and the like. Thus, in the case of a drift between patient 3D temperature estimations and acquired scalp temperature measurements greater than an acceptable drift, then a clinical practitioner is required to investigate the source of the drift.

The computer database 111 storing the benchmark temperature information can further include a benchmark 3D temperature estimation mapping collection 124 of benchmark 3D temperature estimation mappings 126. The benchmark 3D temperature estimation mappings 126 can be determined at different times of the three phases of an induced systemic or local brain hypothermia from cooling from normal core body temperature through to steady state hypothermia, maintaining steady state hypothermia and warming from steady state hypothermia to normal core body temperature. A clinical practitioner can use the benchmark 3D temperature estimation mappings as a clinical reference during an ongoing induced hypothermia and restoring a patient's brain to normal core body temperature.

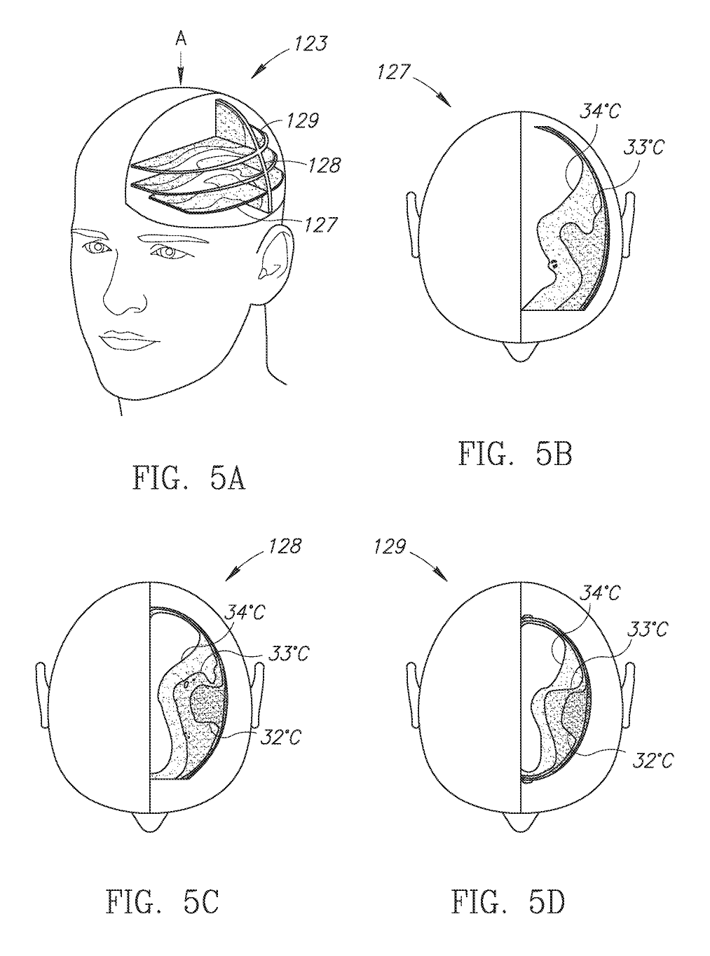

FIG. 5A shows the patient 3D temperature estimation mapping 123 and FIGS. 5B to 5D show three spaced apart transverse cross sections 127, 128 and 129 as follows: FIG. 5B shows the lowermost transverse cross section 127 at a section of the patient's mid-brain. FIG. 5C shows the middle transverse cross section 128 at the patient's level of body of corpus callosum. FIG. 5D shows the uppermost transverse cross section 129 at the patient's superior sagittal sinus (SSS). The indwelling catheter is delivering cold saline downstream of the lowermost transverse cross section 127 and upstream of the middle transverse cross section such that the latter 128 displays a greater chilling effect than the former 127 as evidenced by the middle transverse cross section 128 having a 32° C. isotherm which is missing from the lowermost transverse cross section 127.

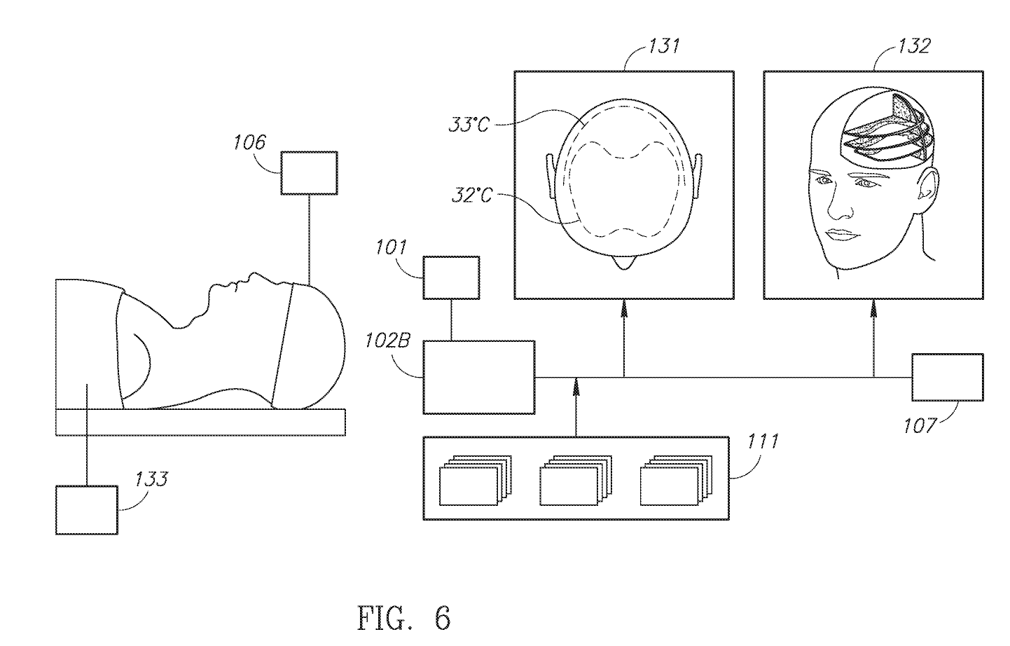

FIG. 6 shows the neuroprotection apparatus 100 including the neuroprotection processor 102B for use during an induced systemic hypothermia for determining real time patient temperature information in the form of a patient scalp isotherm map 131 and/or a patient 3D temperature estimation mapping 132 for display on a human head image on a display device during the induced systemic hypothermia. The neuroprotection apparatus 100 can be equally employed from inception of induced systemic hypothermia through to steady state hypothermia, maintaining steady state hypothermia, and for warming a hypothermic brain from steady state hypothermia through to normal core body temperature The neuroprotection processor 102B processes the chilled blood from, say, a heart lung machine 133 and acquired scalp temperature measurements. The chilled blood is delivered to a patient's head from his right pair of a carotid artery and a vertebral artery and his left pair of a carotid artery and a vertebral artery. FIG. 6 shows that induced systemic hypothermia chills equally both sides of a patient's head as opposed to induced local brain hypothermia which typically chills one side of a patient's head more than his other side.

### Scalp Temperature Measurement Acquisition Devices

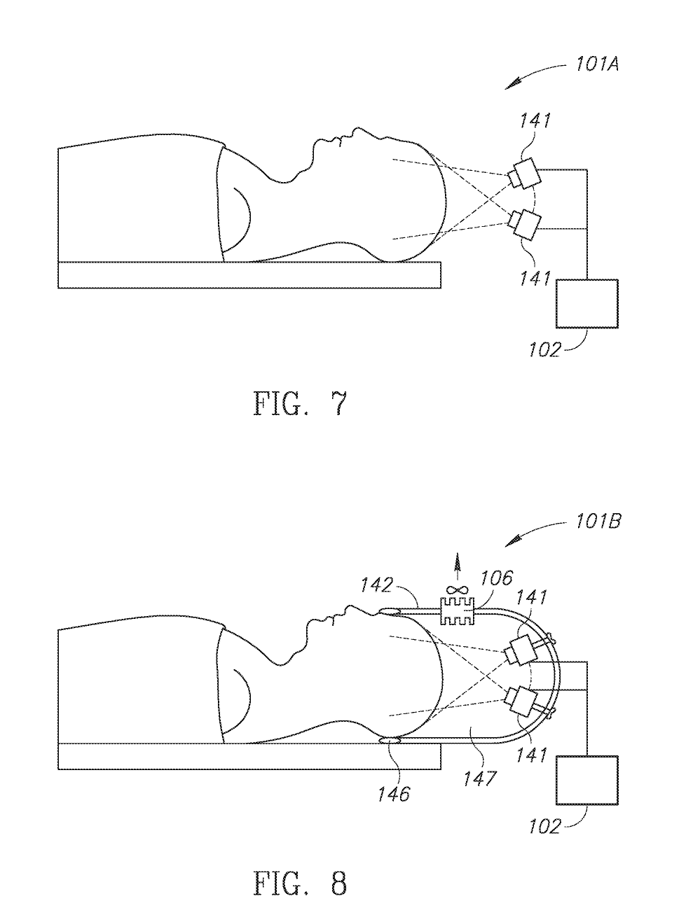

FIG. 7 shows a scalp temperature measurement acquisition device 101A implemented as one or more thermal imaging cameras 141 for acquiring scalp temperature measurements of the patient's scalp. The thermal imaging cameras 141 are preferably arranged such that acquired scalp temperature measurements at a particular location of a patient's scalp can be calculated as the average of two or more scalp temperature measurements from different thermal imaging cameras 141. The thermal imaging cameras 141 preferably have a temperature resolution of 50 mili Kelvin and more than 50,000 pixels in the temperature range of 25° C. to 40° C. and work in the infrared wave length spectrum of 7.5 to 14 μm. The thermal imaging cameras preferably have a dynamic temperature map refresh rate of about 10 refreshes per second. Suitable thermal imaging cameras 141 include inter alia ThermApp thermal cameras available from Opgal www.opgal.com. The thermal imaging cameras 141 each include an optical lens with a short focal length of about 20-30 cm for imaging a patient's scalp.

FIG. 8 shows a scalp temperature measurement acquisition device 101B implemented as an open topped rigid head covering 142 having a base surface 143 and an elongated peripheral surface 144 with a rim 146 for placing on a patient's head such that the head covering 142 bounds a cavity 147 accommodating a patient's scalp. The covering 142 is preferably made from a thermal isolation material such as polycarbonate with a 5-10 millimeter layer of polystyrene foam. The head covering 142 includes one or more thermal imaging cameras 141 similar to the scalp temperature measurement acquisition device 101A. The head covering 142 optionally includes a scalp cooling arrangement 106 for pumping a heat Q from the cavity 147 for cooling a patient's scalp. Scalp cooling arrangement 106 can be optionally implemented by a thermoelectric air-to-air module.

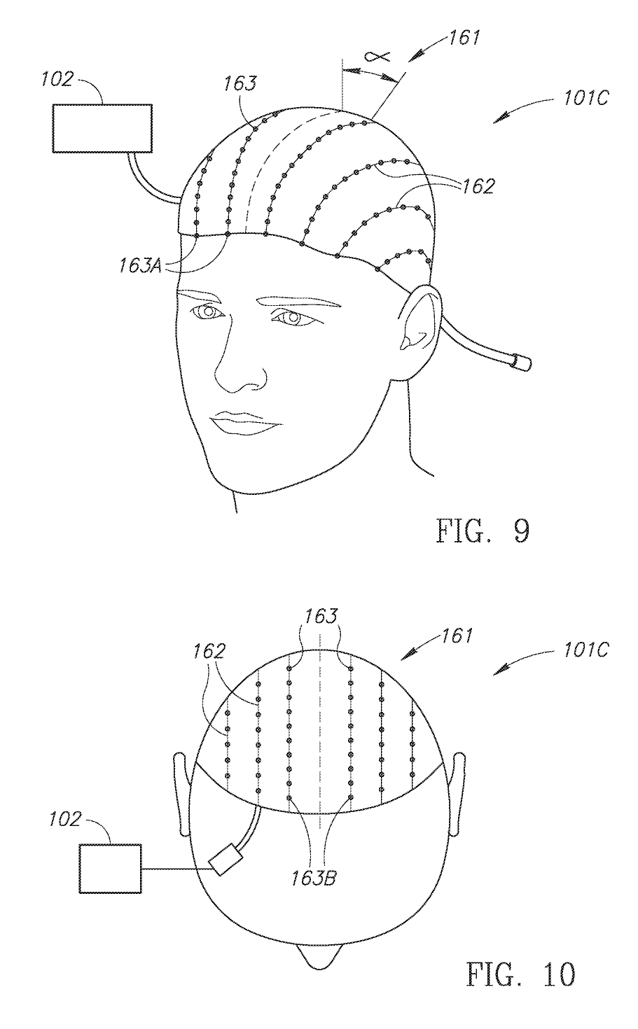

FIGS. 9 and 10 show a scalp temperature measurement acquisition device 101C resembling a stretch fit swim cap made from thermal isolation material, for example, a combination of rubber and polystyrene foam. The scalp temperature measurement acquisition 10 device 101C includes a temperature measurement sensor array 161 in skin contact with a patient's scalp for acquiring scalp temperature measurements. Suitable temperature measurement sensors include inter alia thermistors, thermocouples, and the like. The scalp temperature measurement acquisition device 101C is preferably a disposable single patient use item. The scalp temperature measurement acquisition device 101C can optionally be provided with a scalp cooling arrangement 106 for external cooling a patient's scalp.

The temperature measurement sensor array 161 includes four temperature measurement sensor strips 162 aligned co-directional with the XZ sagittal plane on opposite sides of the patient's head. The preferred separation angle between adjacent temperature measurement sensor strips 162 is about α=15-20°. Each temperature measurement sensor strip 162 includes a 20 foremost sensor 163A above the patient's forehead frontal bone and a rearmost sensor 163B located slightly above the patient's parietal bone. The distance between adjacent sensors 163 along a temperature measurement sensor strip 162 is between about 5 mm and 15 mm such that each temperature measurement strip includes about 25 sensors. Accordingly, the scalp temperature measurement acquisition device 101C includes about 25×8=200 sensors 163. The sensors 163 preferably project between about 0.5 mm and 1.5 mm from the internal surface of the scalp temperature measurement acquisition device 101C for facilitating intimate contact with an outermost scalp skin surface 13. Suitable sensors 163 include, for example, NCP Series thermistors commercially available from Murata Ltd, Japan. Biocompatible thermal conductive gel or adhesive material is preferably employed for facilitating thermal measurements of the outermost scalp skin surface 14.

### Clinical Set-Up for Determining and Administering IACI Regimes

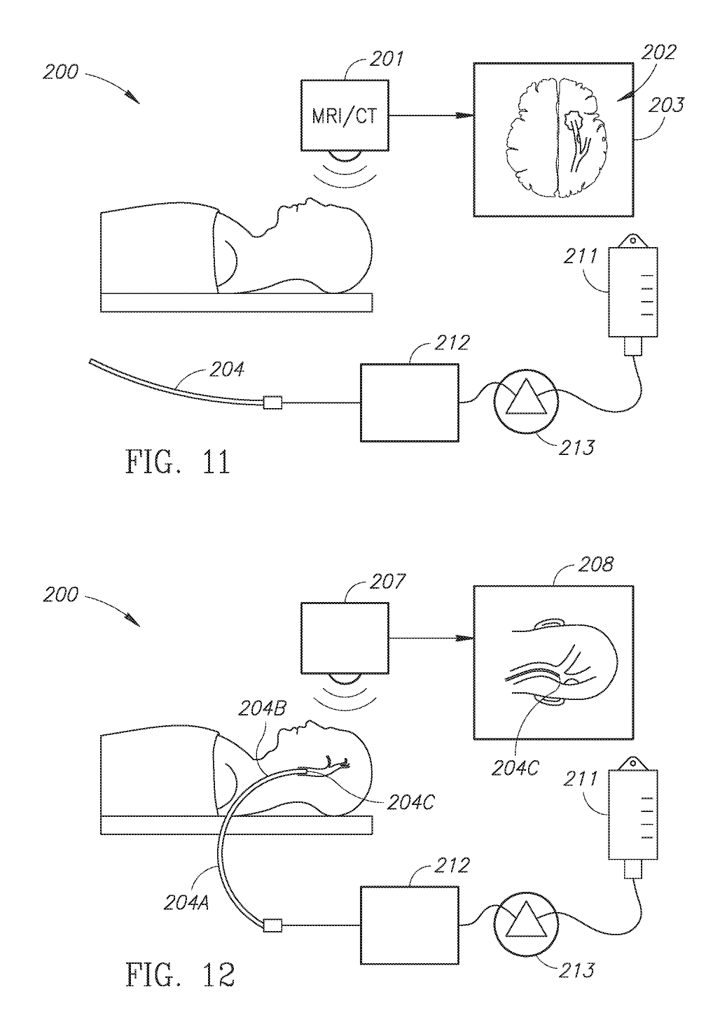

FIGS. 11 and 12 show a clinical set up 200 for determining and administering IACI regimes to stroke brains to minimize additional infarction of penumbra regions.

The clinical set up 200 includes a scan unit 201 for scanning a stroke patient's head 10 for displaying brain scans 202 on a display screen 203. The brain scans 202 can be along XY transverse planes, XZ sagittal planes, YZ coronal planes, and additional planes selected by a clinical practitioner. Suitable scan units 201 include MRI scan units and CT scan units. MRI employs lack of diffusion for determining the three dimensional boundary of an ischemic infarct 10 and perfusion for determining the three dimensional boundary of its associated upstream penumbra region. CT employs perfusion and angiography for determining the three dimensional boundaries of an ischemic infarct and its associated upstream penumbra region. The scan unit 201 is capable of providing brain scans for clearly visualizing a cerebral circulatory structure.

Brain scans 202 provide initial clinical stroke information including inter alia the location of an occlusion and its occlusion percentage, the boundary of its resulting ischemic infarct, the boundary of its associated upstream penumbra region, and the like. A clinical practitioner determines a preferred cerebral target location of an indwelling catheter 204 based on the brain scans 202 to best induce therapeutic hypothermia in the penumbra region 23 in order to prevent further infarction.

Suitable indwelling catheters 204 include inter alia a cerebral angiography diagnostic catheter with a size of 3 to 5 French. An indwelling catheter 204 is typically introduced via a sheath introducer (not shown). Suitable angiography diagnostic catheters include inter alia the Performa series Simmons 2 catheter available from Merit Medical Systems, Inc., Utah, USA www.meritoem.com. Such indwelling catheters 204 include a proximal end 204A for remaining 25 external to a patient and a distal end 204B for placement at a cerebral target location. The distal end 204B includes an opaque marking 204C for X ray imaging purposes. Such indwelling catheters 204 include steerage means for enabling its navigation to a preferred cerebral target location. Such indwelling catheters 204 preferably include insulation to avoid heating of cold saline from its proximal end external to the stroke patient to its distal end at a preferred cerebral target location. The indwelling catheters 204 can optionally be an open irrigation cryocatheter as disclosed in PCT International Application Number PCT/IL2013/050363 entitled Cryocatheter with Coolant Fluid Cooled Thermoelectric Module published under WIPO International Publication Number WO 2013/164820.

The clinical set-up 200 includes a fluoroscopy unit 207 for assisting clinical practitioners to place indwelling catheters 204 at preferred cerebral target locations. The fluoroscopy unit 207 displays a brain image 208 of a stroke brain 12 and the distal end 204B with the opaque marking 204C on a display screen 209. The clinical set-up 200 includes a saline source 211 of cold saline and control apparatus 212 for controlling the administration of an IACI regime to a stroke patient. The control apparatus 212 includes a peristaltic pump 213 for administering cold saline to a stroke patient.

### Benchmark Stroke Brain Clinical Images and Benchmark Scalp Isotherm Maps

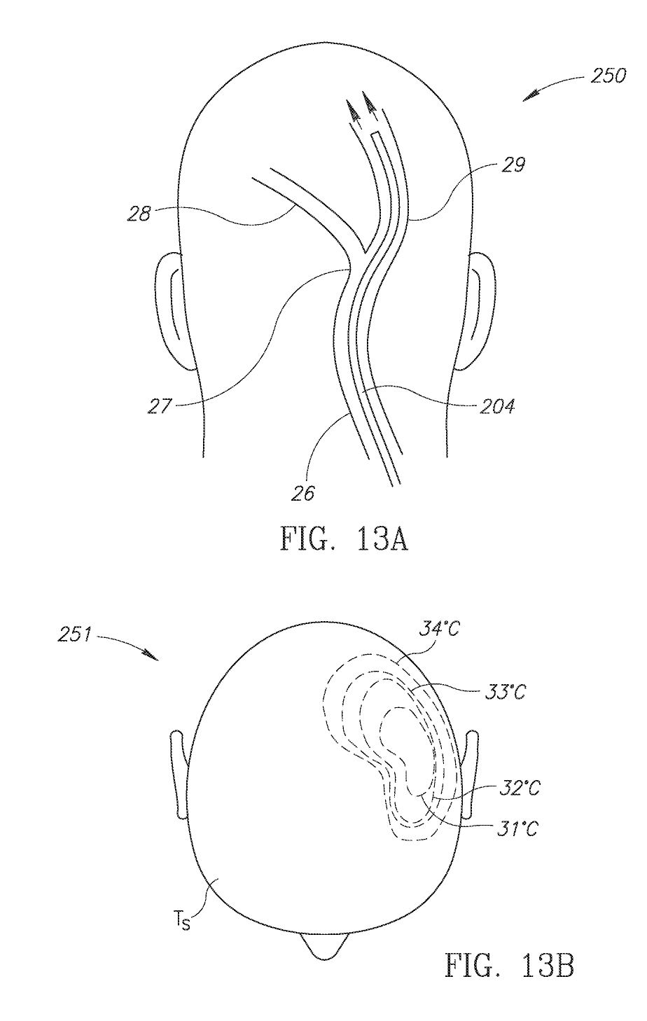

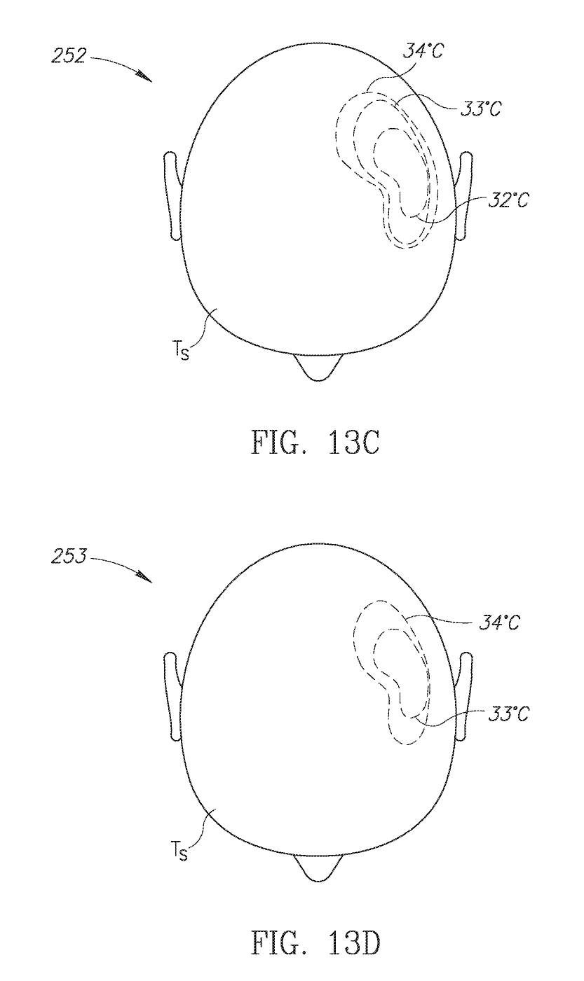

FIGS. 13A, 14A, 15A, 16A and 17A show an Internal Carotid Artery (ICA) 26 bifurcating at a junction 27 into an Anterior Cerebral Artery (ACA) 28 and Middle Cerebral Artery (MCA) 29. FIGS. 13A, 14A, 15A, 16A and 17A show placements of indwelling 15 catheters 204 pushed along the ICA 26 into a proximate section of the MCA 29. FIGS. 13B, 13C, 13D, 14B, 15B, 16C and 17D show scalp isotherm maps at hypothermic steady state.

FIG. 13A shows a placement 250 of an indwelling catheter 204 in a non-stroke brain for providing an incoming flow of chilled saline.

FIG. 13B shows a resulting patient scalp isotherm map 251 determined from the scalp temperature measurements acquired on the patient's scalp for a 15 cc/min flow rate of a 12° C. saline infusion. The patient scalp isotherm map 251 has an innermost 31° C. scalp isotherm showing the skin scalp temperature ranges from a chilled 31° C. to the normal scalp skin temperature Ts.

FIG. 13C shows a resulting patient scalp isotherm map 252 determined from the scalp temperature measurements acquired on the patient's scalp for a 10 cc/min flow rate of a 12° C. saline infusion. The patient scalp isotherm map 252 has an innermost 32° C. scalp isotherm showing the skin scalp temperature ranges from a chilled 32° C. to the normal scalp skin temperature Ts. Comparison of the patient scalp isotherm map 252 to the patient scalp isotherm map 251 shows that the lower flow rate causes a predictable less chilling.

FIG. 13D shows a resulting patient scalp isotherm map 253 determined from the scalp temperature measurements acquired on the patient's scalp for a 10 cc/min flow rate of a 18° C. saline infusion. The patient scalp isotherm map 253 has an innermost 33° C. scalp isotherm showing the skin scalp temperature ranges from a chilled 33° C. to the normal scalp skin temperature Ts. Comparison of the patient scalp isotherm map 253 to the vscalp isotherm map 252 shows that the higher saline infusion temperature causes a predictable less chilling.

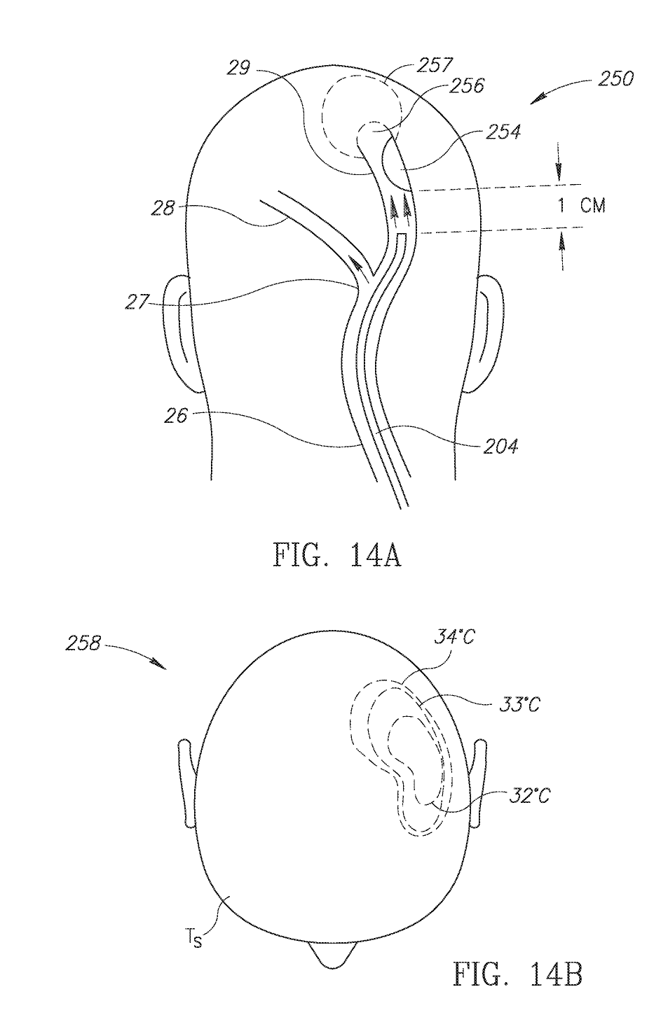

FIG. 14A shows a 70% occlusion 254 along the MCA 29 downstream of the same placement 250 of the indwelling catheter 204 by, say, about 1 cm. The occlusion 254 leads to an infarct 256 and a downstream penumbra region 257. FIG. 14A shows the 70% occlusion 254 diverts a minor proportion of an incoming flow of cold saline from the MCA 29 to the ACA 28 10 to other parts of the brain from the intended downstream penumbra region 257. FIG. 14B shows a resulting patient scalp isotherm map 258 determined from the scalp temperature measurements acquired on the patient's scalp. The scalp isotherm map 258 has an innermost 32° scalp isotherm showing the skin scalp temperature ranges from a chilled 32° to the normal scalp skin temperature Ts.

Comparison between the FIG. 14B patient scalp isotherm map 258 and the FIG. 13B patient scalp isotherm map 251 shows the patient scalp isotherm map 258 does not have a 31° isotherm demonstrating that more of the chilling effect of the cold infusion is dispersed in other areas of the stroke brain not assisting in the required cooling of the penumbra region 257.

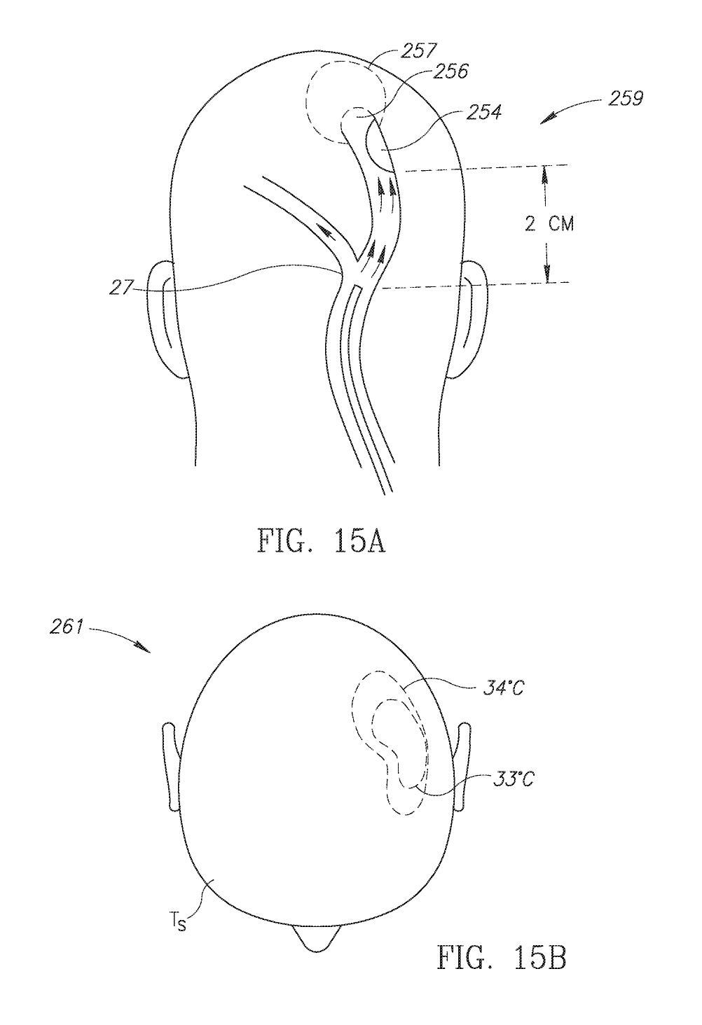

FIG. 15A shows a placement 259 of an indwelling catheter 204 upstream to the 70% occlusion 254 by, say, 2 cm. FIG. 15A shows the placement of the indwelling catheter 204 closer towards the junction 27 leads to considerable more diversion of an incoming flow of cold saline from the penumbra region 257 than compared to FIG. 14A's placement 250. FIG. 15B shows a resulting patient scalp isotherm map 261 determined from the scalp temperature measurements acquired on the patient's scalp. The patient scalp isotherm map 261 has an innermost 33° scalp isotherm showing the skin scalp temperature ranges from a chilled 33° to the normal scalp skin temperature Ts. The lessening of the chilling effect of the IACI regime at the outermost scalp skin surface 14 is due to the major diversion of the chilled saline from the intended penumbra region 257.

Comparison between FIG. 15B and FIG. 14B shows that FIG. 14A's placement 250 of the indwelling catheter 204 is more effective in preventing additional infarction in the penumbra region 257 than FIG. 15A's placement 259 of the indwelling catheter 204. Accordingly, the computer database 111 preferably includes a benchmark stroke brain clinical image 112 and a benchmark scalp isotherm map 113 based on FIGS. 14A and 14B than FIGS. 15A and 15B.

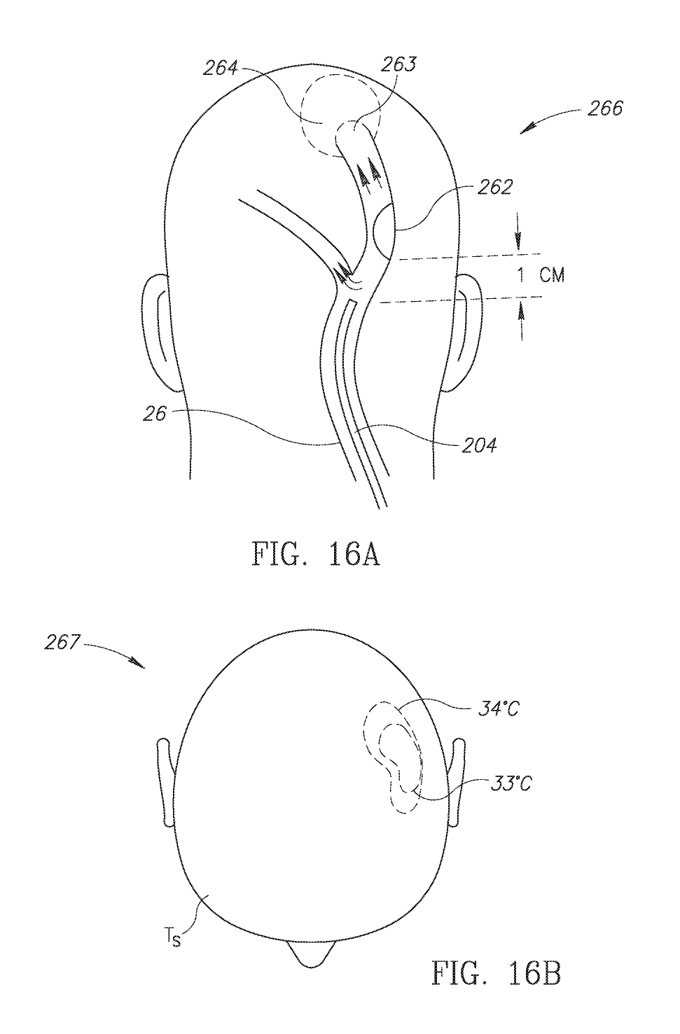

FIG. 16A shows a 90% occlusion 262 leading to an infarct 263 and an associated downstream penumbra region 264. The 90% occlusion 262 typically leads to a larger penumbra region 264 than the 70% occlusion 254's penumbra region 257. FIG. 16A shows a placement 266 of an indwelling catheter 204 for providing an incoming flow of chilled saline for chilling the penumbra region 264. FIG. 16A shows the occlusion 262 diverts the majority of incoming chilled saline from flowing downstream to chill the penumbra region 264 compared to FIGS. 10 14A and 15A.

FIG. 16B shows a resulting patient scalp isotherm map 267 determined from scalp temperature measurements acquired on the patient's scalp. The patient scalp isotherm map 267 has an innermost 33° scalp isotherm showing the skin scalp temperature ranges from a chilled 33° to the normal scalp skin temperature Ts. Comparison between the patient scalp isotherm map 267 and the patient scalp isotherm map 258 shows the patient scalp isotherm map 267 has a smaller 33° scalp isotherm than the patient scalp isotherm map 258 demonstrating that more of the chilling effect of the cold infusion is dispersed in other areas of the stroke brain not assisting in the required cooling of the penumbra region 264.

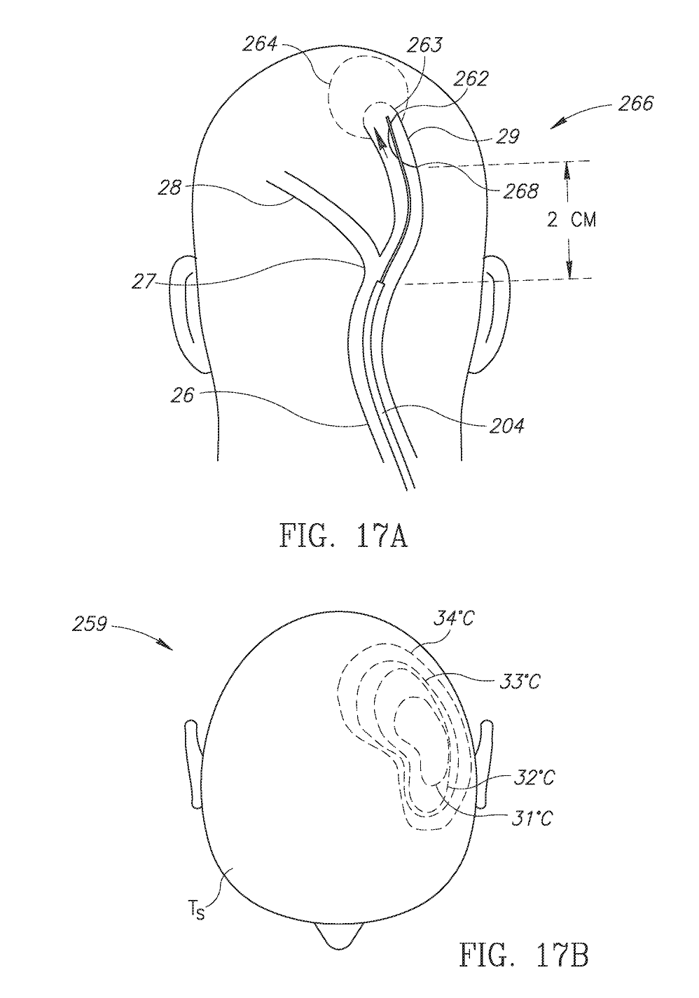

FIG. 17A shows the same placement 266 of the indwelling catheter 204 as FIG. 16A and the use of a micro-catheter 268 for traversing the 90% occlusion 262 for delivering cold saline upstream therefrom by say, 1 cm. FIG. 17B shows a resulting patient scalp isotherm map 269 acquired from the patient's scalp. FIG. 17B shows the beneficial effect of the micro-catheter 268 for preventing additional infarction in the penumbra region 264 in comparison to the patient scalp isotherm map 267.

### Method of Administering an IACI Regime to a Stroke Brain

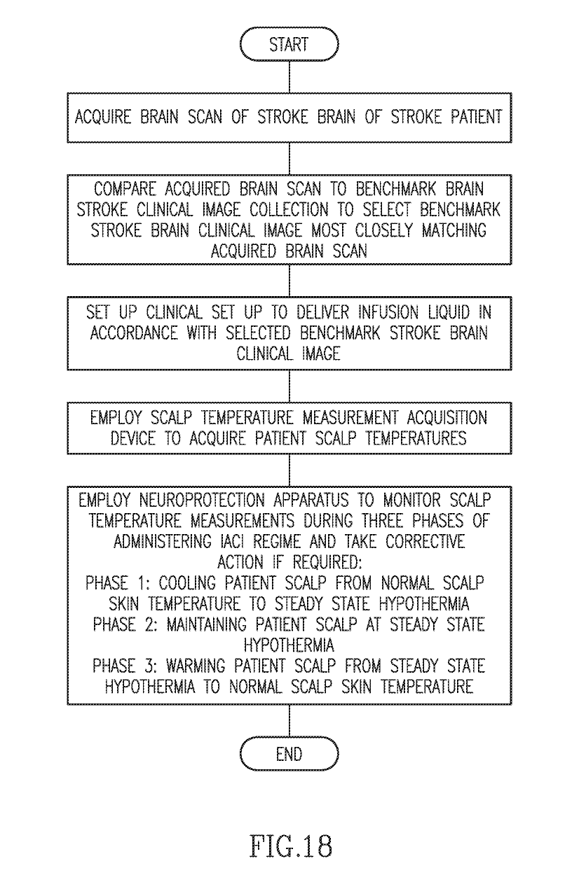

FIG. 18 is a flow diagram showing the use of neuroprotection apparatus 100 for assisting administering an IACI regime to a stroke patient. The use includes inter alia the following steps:

Step 1: Clinical practitioner acquires a brain scan of a stroke brain.

Step 2: Clinical practitioner compares the acquired brain scan to the benchmark stroke brain clinical image collection to select the benchmark stroke brain clinical image mostly closely matching the acquired brain scan.

Step 3: Clinical practitioner inserts an indwelling catheter at a preferred cerebral target for administering an IACI regime to the stroke brain in accordance with the selected benchmark stroke brain clinical image.

Step 4: Clinical practitioner sets the clinical set up to deliver the infusion liquid at a predetermined temperature of the infusion liquid and a predetermined flow rate.

Step 5: Clinical practitioner sets up the neuroprotection apparatus to acquire patient scalp temperature measurements.

Step 6: Clinical practitioner employs the neuroprotection apparatus to monitor the three phases of IACI regime and take corrective if required:

Phase 1: Cooling patient scalp from normal scalp skin temperature Ts to steady state hypothermia.

Phase 2: Maintaining patient scalp at steady state hypothermia

Phase 3: Warming patient scalp from steady state hypothermia to normal scalp skin temperature Ts.

In the case of the neuroprotection processor 102A and the benchmark scalp isotherm map collection 114, a clinical practitioner compares acquired patient scalp isotherm maps to benchmark scalp isotherm maps corresponding to the benchmark stroke brain clinical image to determine whether the acquired patient scalp isotherm map is within an acceptable tolerance. In the affirmative, the clinical practitioner is not required to take any corrective action. In the negative, the clinical practitioner should take corrective action with the intention of reducing the difference between acquired patient scalp isotherm map and the benchmark scalp isotherm map. Such corrective change can include one or more of moving the indwelling catheter, changing the infusion liquid temperature, and changing the infusion liquid flow rate.

For example, if an acquired brain scan is similar to FIG. 14A's benchmark stroke brain clinical image and a clinical practitioner inadvertently places an indwelling catheter in accordance with FIG. 15A's placement 259 rather than FIG. 14A's placement 250, then the resulting patient scalp isotherm map is similar to FIG. 15B's resulting patient scalp isotherm map 261. Accordingly, the clinical practitioner would take corrective action to move the indwelling catheter to FIG. 14A's placement 250 such that the resulting patient scalp isotherm map would largely match FIG. 14B's patient scalp isotherm map 258.

Clinical practitioners can employ the neuroprotection processor 102B and the benchmark 3D temperature estimation mapping collection 124 of benchmark 3D temperature estimation mappings 126 in a similar manner.

While the invention has been described with respect to a limited number of embodiments, it will be appreciated that many variations, modifications, and other applications of the invention can be made within the scope of the appended claims.

1. Neuroprotection apparatus comprising:
(a) a scalp temperature measurement acquisition device for acquiring scalp temperature measurements at a plurality of locations on a patient scalp during an induced hypothermia; and
(b) a neuroprotection processor for processing said scalp temperature measurements for determining real time patient temperature information for display on a human head image on a display device during said induced hypothermia.

2. Apparatus according to claim 1 wherein said neuroprotection processor processes said scalp temperature measurements for determining a patient scalp isotherm map for display on said human head image of said display device.

3. Apparatus according to claim 1 wherein said neuroprotection processor runs a 3D thermal model of an upper generally hemispherical section of a human head for processing flow parameters of an induced hypothermia procedure for determining a patient 3D temperature estimation mapping of a upper generally hemispherical section of a patient head wherein said flow parameters of an induced hypothermia procedure include a location of an indwelling catheter delivering a cold infusion liquid to a patient's brain.

4. Apparatus according to claim 3 wherein said neuroprotection processor includes a 3D thermal model configuration mode for configuring the 3D thermal model including said location of said indwelling catheter therein to an induced hypothermia procedure,
said neuroprotection processor comparing said patient 3D temperature estimation mapping on the patient's scalp to said acquired scalp temperature measurements for determining a deviation index and comparing said deviation index to a deviation threshold for determining whether to re-position said location of said indwelling catheter in said 3D thermal model.

5. Apparatus according to claim 3 wherein said neuroprotection processor superimposes said 3D temperature estimation mapping on a MRI/CT scan image of a patient head constituting said human head image.

6. Apparatus according to claim 1 wherein said neuroprotection processor includes benchmark temperature information and said neuroprotection apparatus compares said patient temperature information to said benchmark temperature information for determining a benchmark error index.

7. Apparatus according to claim 1 wherein said neuroprotection processor includes benchmark temperature information and said neuroprotection processor superimposes said patient temperature information on said benchmark temperature information on said human head image on said display device.

8. Apparatus according to claim 1 wherein said scalp temperature measurement acquisition device includes at least one thermal imaging camera for acquiring said scalp temperature measurements.

9. Apparatus according to claim 1 wherein said scalp temperature measurement acquisition device includes a temperature measurement sensor array in skin contact with the patient scalp.

10. Apparatus according to claim 9 wherein said temperature measurement sensor array includes at least two temperature measurement sensor strips on each side of a sagittal plane of a patient head and co-directional therewith.

11. Apparatus according to claim 9 wherein a said temperature measurement sensor strip includes at least a foremost sensor above a patient forehead frontal bone and a rearmost sensor located slightly above the patient parietal bone.

12. Apparatus according to claim 9 wherein said scalp temperature measurement acquisition device resembles a stretch fit swim cap.

13. Apparatus according to claim 1 wherein said scalp temperature measurement acquisition device further comprises a scalp cooling arrangement for external cooling of a patient scalp.

14. A scalp temperature measurement acquisition device for use with neuroprotection apparatus according to claim 1.

15. A computer database for use with neuroprotection apparatus according to claim 1, the computer database comprising a benchmark stroke brain clinical image collection of benchmark stroke brain clinical images wherein each benchmark stroke brain clinical image includes clinical instructions including at least one of: a placement of an indwelling catheter, infusion liquid flow rate, infusion liquid temperature and infusion liquid duration for administrating the infusion liquid.
